(19) United States
(12) Patent Application Publication
(10) Pub. No.: US 2008/0294010 A1
(43) Pub. Date: Nov. 27, 2008

(54) LARYNGOSCOPE THAT INDICATES CONTACT

(76) Inventor: John D. COOPER, Columbia, MD (US)

Correspondence Address: SUGHRUE MION, PLLC, 2100 PENNSYLVANIA AVENUE, N.W., SUITE 800, WASHINGTON, DC, 20037, US

(21) Appl. No.: 12/126,218

(22) Filed: May 23, 2008

Related U.S. Application Data
(60) Provisional application No. 60/940,161, filed on May 25, 2007.

(51) Int. Cl. A61B 1/267

(52) U.S. Cl. 600/199

(57) ABSTRACT

A laryngoscope including a laryngoscope blade and a detector incorporated in or attached to the laryngoscope blade, the detector detecting contact. A method of using a laryngoscope including moving a patient's pharyngeal structure using a laryngoscope blade, wherein a detector is attached to the laryngoscope blade; contacting the patient's teeth with the detector; and indicating a contact between the detector and the teeth.

This application claims priority from U.S. provisional application No. 60/940,161, which was filed on May 25, 2007, the entire contents of which are hereby incorporated by reference.

### BACKGROUND OF THE INVENTION

1. Field of the Invention

Devices and methods and consistent with the present invention relate to a laryngoscope and the use thereof and more specifically to a laryngoscope which indicates dental contact for the protect of teeth during the laryngeal intubation process and the use thereof.

2. Description of the Related Art

Intubation generally refers to a process by which a tube is placed into an external or internal orifice of the body. However, intubation most often refers to the laryngeal intubation process, in which a tube is placed in a patient's trachea. A laryngoscope with a laryngoscope blade has been used to assist with laryngeal intubation for many years. Typically, an end of a laryngoscope blade is inserted into the patient's open mouth, and the laryngoscope blade is used to move the pharyngeal structures, allowing for intubation of the trachea.

Specifically, the process of intubation requires the insertion of the long laryngoscope blade into the patient's mouth, as shown in FIG. 1. The laryngoscope blade then is manipulated by the user to move the soft tissue pharyngeal structures, such as the tongue or epiglottis. This provides a view of the glottis and an opening in which an endotracheal tube is placed. That is, by moving these soft tissue pharyngeal structures, the structures are prevented from blocking the alignment of the trachea with the oral axis, allowing for endotracheal intubation.

However, conventional laryngoscope blades do not protect the patient's teeth during intubation. That is, in the process of manipulating the laryngoscope blade, the laryngoscope blade can inadvertently come in contact with the patient's teeth. The levering forces of the laryngoscope placed on the patient's teeth can cause damage to or displacement of the teeth. Moreover, conventional laryngoscope blades cannot provide any warning of this impending dental damage.

Thus, while conventional laryngoscope devices may be suitable for providing intubation, they do not suitably protect the patient's teeth during the laryngeal intubation process. For example, because many laryngoscope blades are made of metal or a high tensile strength plastic, inadvertent contact by the laryngoscope blade with the patient's teeth could cause damage or displacement of the teeth.

Therefore, it would be beneficial to provide a device and method that warns the user of the laryngoscope of this contact so that damage to the teeth, and related morbidity, can be avoided. In these respects, a laryngoscope which indicates dental contact would protect the patient's teeth during the laryngeal intubation process.

### SUMMARY OF THE INVENTION

According to a first aspect of the invention, a laryngoscope includes a laryngoscope blade and a detector incorporated in or attached to the laryngoscope blade, the detector detecting contact.

The detector can include a switch, such as a membrane switch. The laryngoscope can also include an indicator that provides an indication of the contact. The indicator can be a visual indicator and/or an auditory indicator.

The laryngoscope can include a sensor body attached to the laryngoscope blade, wherein the sensor body includes the detector and the indicator. In addition, the laryngoscope can include a handle attached to the laryngoscope blade, the handle being provided opposite the sensor body, and a light source that provides light in substantially a longitudinal direction of the laryngoscope blade. The light source can be attached to the laryngoscope blade.

A battery that provides power to the indicator can be can provided within the sensor body or provided within the handle.

The laryngoscope can include a video camera that provides a view of an inside of a patient's mouth on a screen, wherein the screen displays a visual indication of the contact.

According to a second aspect of the invention, a method of using a laryngoscope includes moving a patient's pharyngeal structure using a laryngoscope blade, wherein a detector is attached to the laryngoscope blade; contacting the patient's teeth with the detector; and indicating a contact between the detector and the teeth.

According to a third aspect of the invention, a laryngoscope includes a laryngoscope blade; a means for detecting contact with a patient's teeth incorporated in or attached to the laryngoscope blade; and a means for indicating contact between the detector and the patient's teeth.

### BRIEF DESCRIPTION OF THE DRAWINGS

The above and/or other aspects of the present invention will become apparent and more readily appreciated from the following description of the exemplary embodiments, taken in conjunction with the accompanying drawings of which:

FIG. 1 shows the insertion of a laryngoscope blade into a patient's mouth;

FIG. 2 is a perspective view of the parts of a first exemplary embodiment of a laryngoscope;

FIG. 3 shows a detector according to the exemplary embodiment;

FIG. 4 shows a sensor body of the exemplary embodiment; and

FIG. 5 is a perspective view of the parts of an exemplary embodiment of a second exemplary embodiment of a laryngoscope.

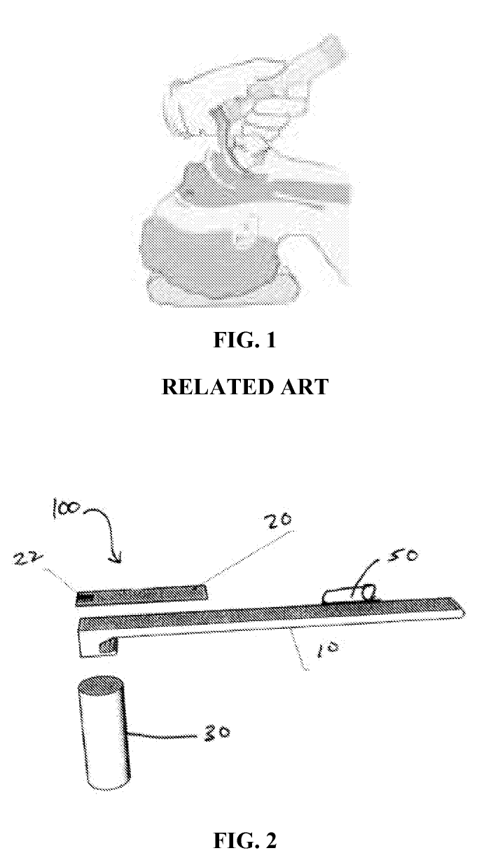

### DETAILED DESCRIPTION OF THE EXEMPLARY EMBODIMENTS

Reference will now be made in detail to an exemplary embodiments of the present invention, examples of which are illustrated in the accompanying drawings, wherein like reference numerals refer to like elements throughout. It is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

A first exemplary embodiment of the laryngoscope is shown in FIG. 2. The laryngoscope 100 generally includes a laryngoscope blade 10, a sensor body 20 attached to the laryngoscope blade 10, and a handle 30. The laryngoscope blade 20 is typically a rigid member made of metal or a high tensile strength plastic and is attached to the handle 30 in a conventional manner. Moreover the laryngoscope blade can include a light source 50 that provides light in substantially a longitudinal direction of the laryngoscope blade. However, the invention is not limited in these respects.

The sensor body 20 can be attached to the laryngoscope blade 10 by, for example, an adhesive. However, the invention is not limited in this respect and the sensor body can be attached in other ways.

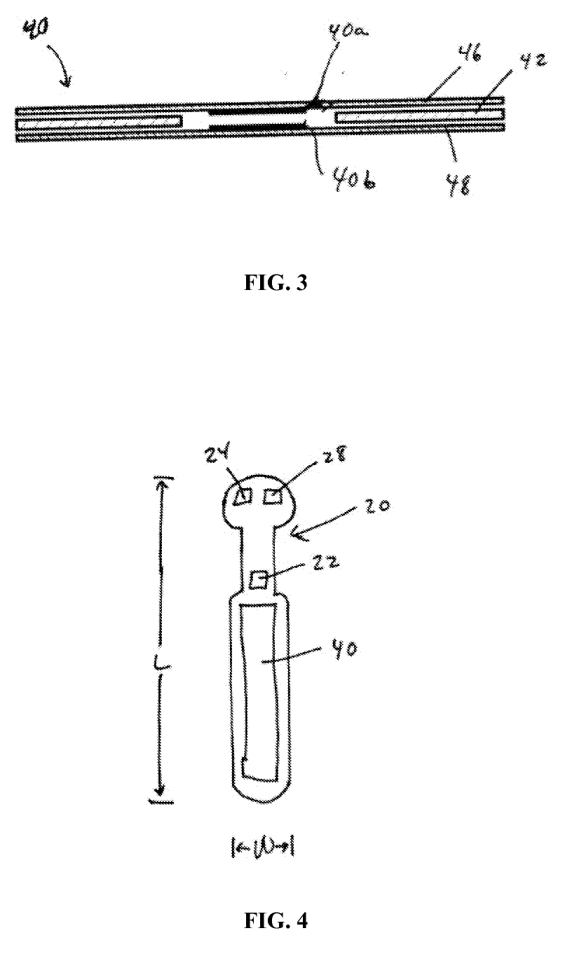

As shown in FIG. 3, the detector 40 can be, for example, a switch including spaced electrical contacts 40a, 40b. When the sensor body 20 contacts the patient's teeth, a circuit including at least one indicator 22, 24 and a power source 28 (shown in FIG. 4) is closed, causing the indicator 22, 24 to provide an indication of the contact.

The detector 40 can be a switch, as shown in FIG. 3. The switch includes a flexible upper layer 46, a first contact 40a provided on the flexible upper layer 46, a spacer 42, a lower layer 48, and a second contact 40b provided on the lower layer 48. The spacer 42 provides a separation between the first and second contacts 40a, 40b when the detector has not been contacted by the patient's teeth. At this state, there is an open circuit and no indication is provided.

The flexible upper layer 46 can be, for example, plastic or rubber. When the upper layer 46 is deformed downward by contact with the patient's teeth, the contacts 40a and 40b close a circuit formed by upper circuitry on the upper layer 46 and lower circuitry on the lower layer 48. This causes the indicator 22, 24 to provide an indication of the contact.

As shown in FIG. 4, this exemplary embodiment includes two indicators, a visual indicator 22 and an auditory indicator 24; however, the invention is not limited in this respect. The visual indicator 22 of the exemplary embodiment shown in FIG. 2 is provided on an outer surface of the sensor body 20. The visual indicator 22 can be a light emitting diode (LED) that is provided on the surface of the sensor body 20 and that emits light when the contact occurs. An LED is a semiconductor device that emits incoherent narrow-spectrum light when electrically biased in the forward direction.

The auditory indicator 24 can be, for example, a piezoelectric device such as a buzzer or tweeter, an audio transducer, or an ultrasonic transducer. The auditory indicator 24 can be directly connected to the contacts 40a, 40b so that an indicator signal of sound waves is transmitted through air when there is contact between the contacts 40a, 40b. Piezoelectricity is the ability of certain crystals to generate a voltage in response to applied mechanical stress. The word is derived from the Greek piezein, which means to squeeze or press. The piezoelectric effect is reversible in that piezoelectric crystals, when subjected to an externally applied voltage, can change shape by a small amount thus producing sound. The possible structure and functions of the piezoelectric buzzer can vary by substance type and sound intensity for indication of dental contact.

However, the invention is not limited by these examples of indicators. For example, the indicator of dental contact could instead be a variation of the emitted light provided by the laryngoscope light source 50. Moreover, an electrical signal provided by the closing of switch's contacts could be sent to another device that provides audio and/or visual indication but is not a part of the laryngoscope.

As shown in FIG. 4, the power source 28 that is used to power the indicator 22, 24 can be a battery, such as a type of battery typically used in watches, that is provided within the sensor body 20. A battery is a device that stores chemical energy and makes it available in an electrical form. Batteries consist of electrochemical devices such as one or more galvanic cells, fuel cells or flow cells.

There can be alternate versions or modifications of the exemplary embodiment that are consistent with the invention. For example, although the detector 40 shown in FIG. 2 is provided within a separate sensor body 20 that is attached to the laryngoscope blade 10, the detector 40 can also be incorporated in the blade 10. In such a modification, the top surface of the detector 40 is also the top surface of the blade 10.

Likewise, the exemplary embodiment discloses the power source 28 also provided within the sensor body 20. However, the invention is not limited in this respect and a power source (not shown), such as a battery, provided within the handle 30 can serve as the power source for the detector 40 and/or indicator 22, 24. This power source within the handle could also provide power for the laryngoscope light source 50.

In addition, the detector 40 can also provide an indication, not only of contact with the patient's teeth, but also of the intensity of the contact with the patient's teeth to prevent damage to the dental structures. For example, the detector 40 can include multiple switches, with an increased visual or auditory indication provided as more switches are closed. Alternatively, a different visual or auditory indication could be used to indicate the intensity of the contact. For example, a change in color of the visual indication or a change in tone of the auditory indication.

The detector 40 can be a membrane switch. A membrane switch is a rectangular device with a variety of layers which can be designed to modify the behavior of the device in a multitude of ways. Therefore, a membrane switch would be especially appropriate to indicate the intensity of the contact.

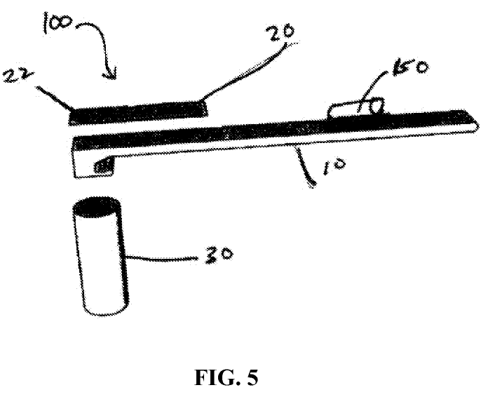

According to a second exemplary embodiment shown in FIG. 5, the laryngoscope 200 still includes a laryngoscope blade 10, a sensor body 20 attached to the laryngoscope blade 10, and a handle 30. However, in this exemplary embodiment, the laryngoscope includes a video camera 150 with a light source instead of only a light source. The video camera 150 is used to provide a view of the inside the patient's mouth on a video screen (not shown). The video screen can be used to display some visual indication of the contact between a detector and the patient's teeth, such as a contact icon or a color change.

As to a further discussion of the manner of usage and operation of the present invention, the same should be apparent from the above description. Accordingly, no further discussion relating to the manner of usage and operation will be provided. With respect to the exemplary embodiments discussed above, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention. For example, although the sensor body 20 shown in FIG. 4 has a length L of 5 to 6 inches, which is about the same length as the laryngoscope blade, and a width W of 0.5 inches, the invention is not limited in this respect.

Therefore, the foregoing exemplary embodiments are considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A laryngoscope, comprising:
a laryngoscope blade; and
a detector incorporated in or attached to the laryngoscope blade, the detector detecting contact.

2. The laryngoscope of claim 1, wherein the detector comprises a switch.

3. The laryngoscope of claim 2, wherein the switch comprises a membrane switch.

4. The laryngoscope of claim 2, further comprising an indicator that provides an indication of the contact.

5. The laryngoscope of claim 4, wherein the indicator comprises at least one of a visual indicator or an auditory indicator.

6. The laryngoscope of claim 5, wherein the indicator is the visual indicator.

7. The laryngoscope of claim 6, wherein the visual indicator is a light emitting diode.

8. The laryngoscope of claim 5, wherein the indicator is the auditory indicator.

9. The laryngoscope of claim 8, wherein the indicator is a piezoelectric device.

10. The laryngoscope of claim 5, wherein the indicator comprises a visual indicator and an auditory indicator.

11. The laryngoscope of claim 4, further comprising a sensor body attached to the laryngoscope blade, wherein the sensor body comprises the detector.

12. The laryngoscope of claim 11, wherein the sensor body further comprises the indicator.

13. The laryngoscope of claim 11, further comprising a handle attached to the laryngoscope blade, the handle provided opposite the sensor body.

14. The laryngoscope of claim 13, further comprising a light source that provides light in substantially a longitudinal direction of the laryngoscope blade.

15. The laryngoscope of claim 14, wherein the light source is attached to the laryngoscope blade.

16. The laryngoscope of claim 15, wherein a battery is provided within the handle; the battery providing power to the light source and the indicator.

17. The laryngoscope of claim 11, further comprising a battery that provides power to the indicator, the battery being provided within the sensor body.

18. The laryngoscope of claim 4, further comprising a video camera that provides a view of an inside of a patient's mouth on a screen, wherein the screen displays a visual indication of the contact.

19. A method of using a laryngoscope, comprising:
moving a patient's pharyngeal structure using a laryngoscope blade, wherein a detector is attached to the laryngoscope blade;
contacting the patient's teeth with the detector; and
indicating a contact between the detector and the teeth.

20. The method of claim 19, wherein the detector comprises a switch.

21. The method of claim 20, wherein the indicating the dental contact comprises at least one of a visual indication or an auditory indication.

22. A laryngoscope, comprising:
a laryngoscope blade;
a means for detecting contact with a patient's teeth incorporated in or attached to the laryngoscope blade; and
a means for indicating contact between the detector and the patient's teeth.
